(19) United States
(12) Patent Application Publication
(10) Pub. No.: US 2017/0000519 A1
(43) Pub. Date: Jan. 5, 2017

(54) INTERVENTIONAL WIRE CAPTURE DEVICE AND METHODS OF USE

(71) Applicant: Furqan Tejani, Yonkers, NY (US)

(72) Inventor: Furqan Tejani, Yonkers, NY (US)

(21) Appl. No.: 14/788,172

(22) Filed: Jun. 30, 2015

(51) Int. Cl. A61B 17/34; A61B 17/3207

(52) U.S. Cl. CPC A61B 17/3403; A61B 17/3207

(57) ABSTRACT

Disclosed herein are methods of traversing a chronic total occlusion (CTO) that include advancing a recanalization wire through the vasculature of the subject through a puncture site in an artery of a lower extremity towards a distal cap of the CTO, advancing the recanalization wire through the CTO until a distal tip of the recanalization wire exits the CTO via a proximal cap of the CTO, magnetically capturing the distal tip of the recanalization wire, pulling the recanalization wire through a puncture side in a femoral artery, thus externalizing the recanalization wire. Some capture wires may include a magnetic tip, such as a neodymium tip.

### TECHNICAL FIELD

Embodiments relate to a wire capture device for vascular interventions, such as traversing a chronic total occlusion (CTO).

### BACKGROUND

Peripheral artery disease (PAD) includes stenosis and occlusion of upper- or lower-extremity arteries due to atherosclerotic or thromboembolic disease. PAD represents a spectrum of disease severity, encompassing both asymptomatic and symptomatic disease. In PAD, as blood vessels narrow, arterial flow into the extremities worsens, and symptoms may manifest either as classic intermittent claudication (IC) or as atypical claudication or leg discomfort. As the disease progresses, patients may develop more severe claudication, with reduced walking distance and eventually with rest pain. In 5-10 percent of cases, claudication progresses to a worsened severity of the disease, called critical limb ischemia (CLI), which is defined as ischemic rest pain for more than 14 days, ulceration, or tissue loss/gangrene. Patients with CLI have a mortality of 25 percent at one year.

Multiple types of interventions are used for revascularization in patients with PAD, including open surgery, angioplasty (e.g., cryoplasty or angioplasty with drug-coated, cutting, or standard angioplasty balloons), stenting (e.g., with self-expanding or balloon-expandable stents), and atherectomy (e.g., using laser, directional, orbital, or rotational atherectomy devices). With improvements in endovascular techniques and equipment, the use of balloon angioplasty, stenting, and atherectomy has led to application of endovascular revascularization to a wider range of patients, both among those with more severe symptoms and those with less severe symptoms. However, such interventions frequently involve first traversing a stenosis with a wire, catheter, or treatment device, which can be difficult to accomplish, due to the fibrous and calcified nature of such stenoses.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIG. 1A illustrates a portion of a peripheral artery with a chronic total occlusion (CTO) therein, and FIG. 1B illustrates a close-up view of the CTO, showing the hardened proximal cap, in accordance with various embodiments;

FIG. 2 illustrates a partial cutaway view of a peripheral artery in which a typical proximal or anterograde approach to traversing a CTO is being performed, and wherein the recanalization wire has entered the sub-intimal space, in accordance with various embodiments;

FIGS. 3A and 3B illustrate the major arteries of the foot (FIG. 3A) and one embodiment of a distal or retrograde approach in which a catheter is advanced retrogradely towards a CTO in the popliteal artery via the dorsalis pedis artery (FIG. 3B);

FIGS. 4A and 4B partial cutaway views of a peripheral artery in which a CTO is being traversed using an embodiment of a distal or retrograde approach, wherein a wire is advanced to the distal cap of the CTO inside a microcatheter (FIG. 4A), and wherein the wire and microcatheter are then advanced together through the CTO (FIG. 4B), in accordance with various embodiments;

FIGS. 5A and 5B illustrate partial cutaway views of a peripheral artery in which a CTO is being traversed using another embodiment of a distal or retrograde approach, wherein a wire is advanced from the distal cap of the CTO through the proximal cap (FIG. 5A), where it is then captured by a capture wire using a neodymium magnet (FIG. 5B), in accordance with various embodiments; and

FIG. 6 illustrates a CTO in the superficial femoral artery, wherein the wire has traversed the CTO and is externalized through a first sheath in the dorsalis pedis branch of the tibial artery and a second sheath in the common femoral artery, in accordance with various embodiments.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “NB” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

Embodiments herein provide interventional wire capture devices and methods for treating peripheral chronic total occlusion (CTO). Conventional percutaneous treatments for CTO typically involve advancing a recanalization wire through an artery, such as the femoral artery, to the periphery until the wire reaches the proximal cap of the CTO. The wire is then advanced through the proximal cap to traverse the occlusion. In some instances, it may be difficult to penetrate the hardened proximal cap because it may be made of dense, fibrous tissue and calcified materials. Thus, when advanced in a proximal direction in this fashion, wires have a tendency to bypass the proximal cap and enter the sub-intimal space during an attempt to recanalize the occlusion.

In the methods disclosed herein, instead of traversing the occlusion in an anterograde direction, the recanalization wire is advanced in a retrograde direction, such that it may enter the occlusion through the distal cap. The proximal cap of a CTO may be dense, fibrous, and highly calcified, but the extent of calcification and fibrous tissue typically is much lower in the distal cap. Therefore, the recanalization wire may more easily penetrate the distal cap, and may traverse the occlusion without entering the sub-intimal space.

Thus, in various embodiments, a sheath may be placed in an artery of the lower leg or foot, such as the popliteal artery or the dorsalis pedis branch of the anterior tibial artery, and a wire may be advanced retrogradely through the anterior tibial artery and/or popliteal artery until the distal cap of the CTO is reached. In various embodiments, the characteristics of a CTO are such that, when approached from a retrograde approach, it is relatively easy to traverse the CTO and recanalize the vessel.

In one specific, non-limiting example, access to the vasculature may be obtained through a puncture site in the foot or lower leg, and a recanalization wire may be advanced retrogradely through the dorsalis pedis branch of the anterior tibial artery or the popliteal artery inside a microcatheter. When the distal cap of the CTO is reached, the microcatheter and recanalization wire may be advanced together through the CTO until they have passed completely through the proximal cap and exited the CTO. Without being bound by theory, it is believed that the microcatheter may provide stability and directional control when traversing the CTO, making it easier to traverse the entire CTO when compared to traversing the CTO with the recanalization wire, alone.

In another specific, non-limiting example, a recanalization wire may be advanced retrogradely through the anterior tibial artery or popliteal artery as described above until the distal cap of the CTO is reached. As described above, the recanalization wire may then be advanced through the CTO (either alone or inside a microcatheter) until the proximal cap of the CTO has been traversed. In various embodiments, the recanalization wire may include a ferromagnetic tip.

Access to the femoral artery may be obtained through a puncture site in the groin, and a capture wire may then be advanced anterogradely through the femoral artery as described above until it reaches the distal ferromagnetic tip of the recanalization wire at the proximal cap of the CTO. In various embodiments, the distal tip of the capture wire may include a neodymium magnet, and thus the distal tip of the capture wire may have a permanent magnetic charge that may have, for example, 10 times the strength of a naturally occurring magnet, or even more. In various embodiments, when the neodymium tip of the capture wire makes contact with the ferromagnetic tip of the recanalization wire, they couple together with a magnetic force having sufficient strength to allow the user to pull the recanalization wire towards the aperture in the femoral artery, thus externalizing the wire. In various embodiments, this may create a complete traversal of the CTO with a wire extending from the sheath in the femoral artery, through the CTO, and exiting through a sheath in the popliteal artery or the dorsalis pedis branch of the anterior tibial artery.

In various embodiments, once the CTO has been completely traversed and the recanalization wire has been externalized through the femoral artery, the user may perform a procedure such as angioplasty or stenting in order to widen the passage through the CTO, thus treating the CTO and restoring blood flow to the limb.

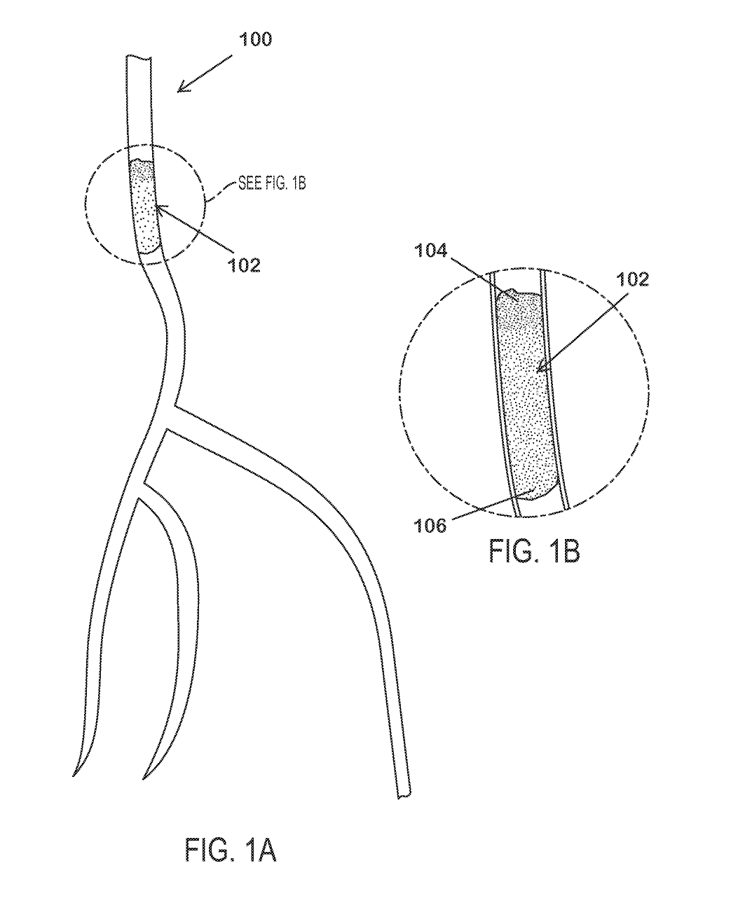

Turning now to the figures, FIG. 1A illustrates a portion of a peripheral artery 100 with a chronic total occlusion (CTO) 102 therein, and FIG. 1B illustrates a close-up view of the CTO 102, showing the hardened proximal cap 104, in accordance with various embodiments. As described above, in some instances, it may be difficult to penetrate the hardened proximal cap 104 because it may be made of dense, fibrous tissue and/or calcified materials. Additionally, in some instances, tortuosities near the CTO may prevent the generation of the high penetration forces that may be necessary in order to traverse a highly calcified proximal cap. By comparison, the distal cap 106 may be considerably less dense, fibrous, and calcified.

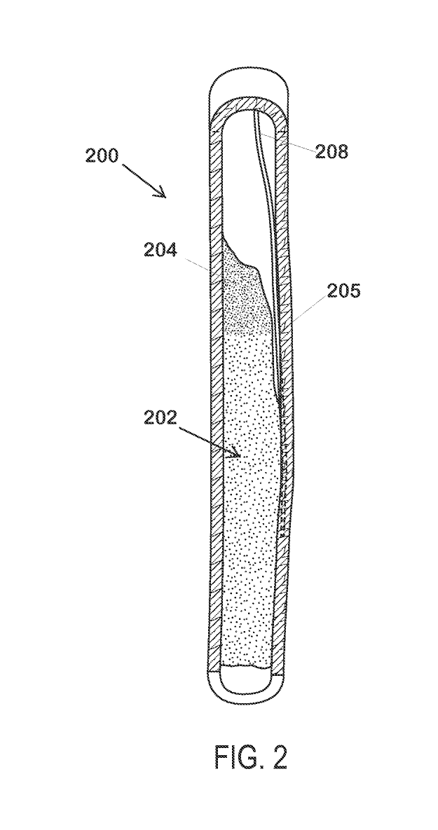

FIG. 2 illustrates a partial cutaway view of a peripheral artery in which a typical proximal or anterograde approach to traversing a CTO is being performed, and wherein the recanalization wire has entered the sub-intimal space, in accordance with various embodiments. Conventional percutaneous treatments for CTO typically involve advancing a recanalization wire 208 anterogradely through an artery 200, such as the femoral artery, to the periphery until the wire reaches the proximal cap 204 of the CTO 202. The wire is then advanced through the proximal cap 204 to traverse the CTO 202. In some instances, it may be difficult to penetrate the hardened proximal cap 204 because it may be made of dense, fibrous tissue and calcified materials. Thus, when advanced in a proximal direction in this fashion, the recanalization wire 208 has a tendency to bypass the proximal cap 204 and enter the sub-intimal space 205 during an attempt to recanalize the occlusion 202.

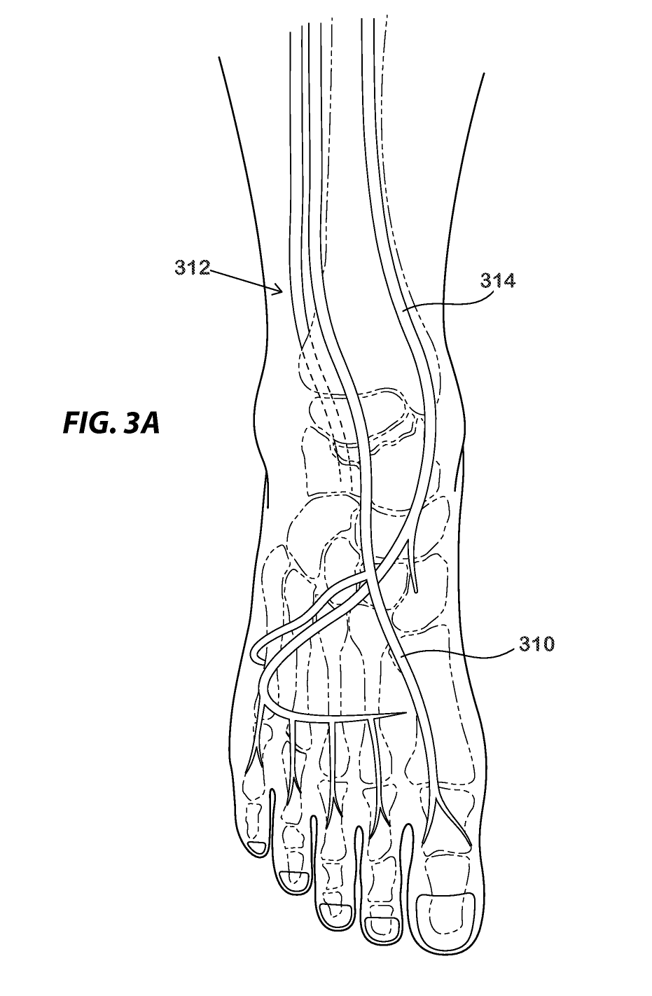

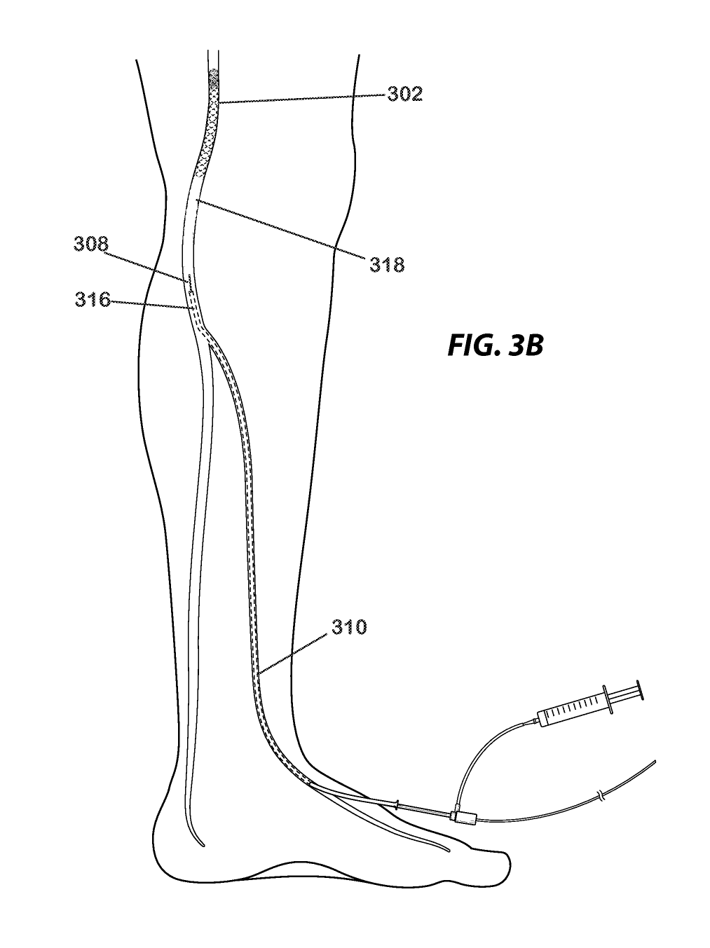

FIGS. 3A and 3B illustrate the major arteries of the foot (FIG. 3A) and an example of a method in which a catheter is advanced retrogradely towards a CTO in the leg via the dorsalis pedis artery (FIG. 3B). As illustrated in FIG. 3A, any of several arteries of the foot and lower leg may be selected for use in the methods disclosed herein, including the popliteal artery, dorsalis pedis 310, the posterior tibialis 312, and the peroneal (fibular) artery 314. FIG. 3B illustrates an example in which a recanalization wire 308 is being advanced inside a microcatheter 316 in a retrograde direction via the dorsalis pedis artery 310 and into the popliteal artery 318, where the CTO 302 is located.

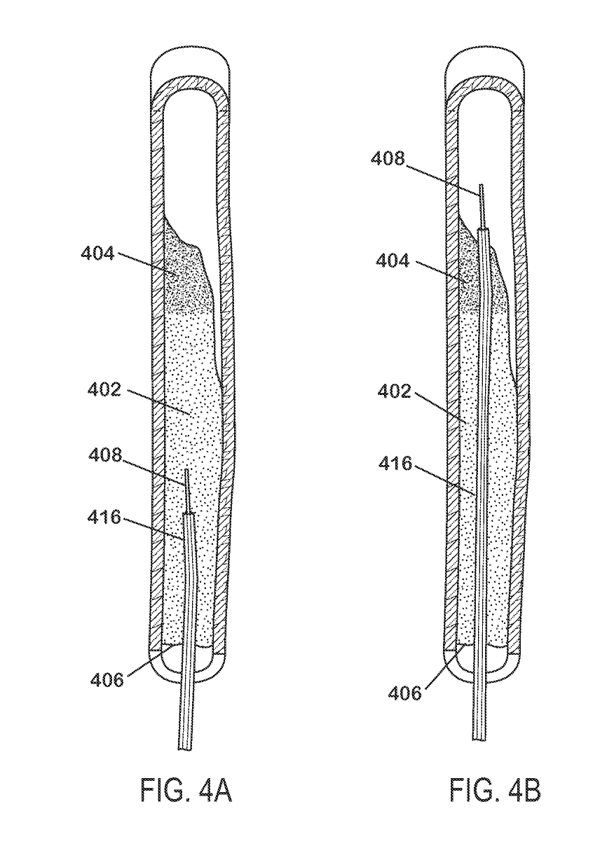

FIGS. 4A and 4B partial cutaway views of a peripheral artery in which one embodiment of a distal or retrograde approach to traversing a CTO is carried out, wherein a wire is advanced to the distal cap of the CTO inside a microcatheter (FIG. 4A), and wherein the wire and microcatheter are then advanced together through the CTO (FIG. 4B), in accordance with various embodiments. In this embodiment, access to the vasculature may be obtained through a puncture site in the foot or lower leg, and a recanalization wire may be advanced retrogradely through the dorsalis pedis branch of the anterior tibial artery or the popliteal artery inside a microcatheter, as illustrated in FIG. 3B. When the distal cap 406 of the CTO 402 is reached, the microcatheter 416 and recanalization wire 408 may be advanced together through the CTO 402 until they have passed completely through the proximal cap 404 and exited the CTO 402. In some embodiments, the microcatheter 416 may provide stability and directional control to the recanalization wire 408 when traversing the CTO 402, making it easier to traverse the entire CTO 402 as compared to traversing the CTO 402 with the recanalization wire 408 alone.

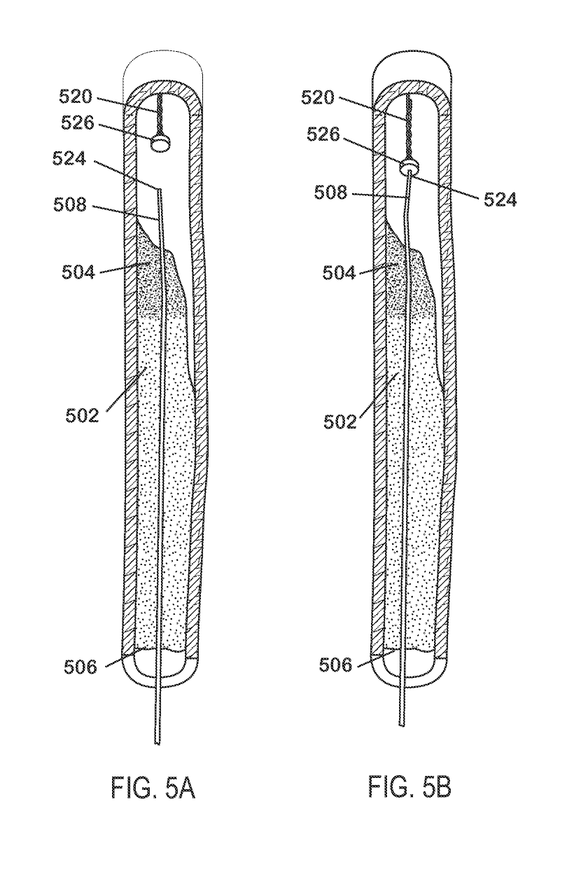

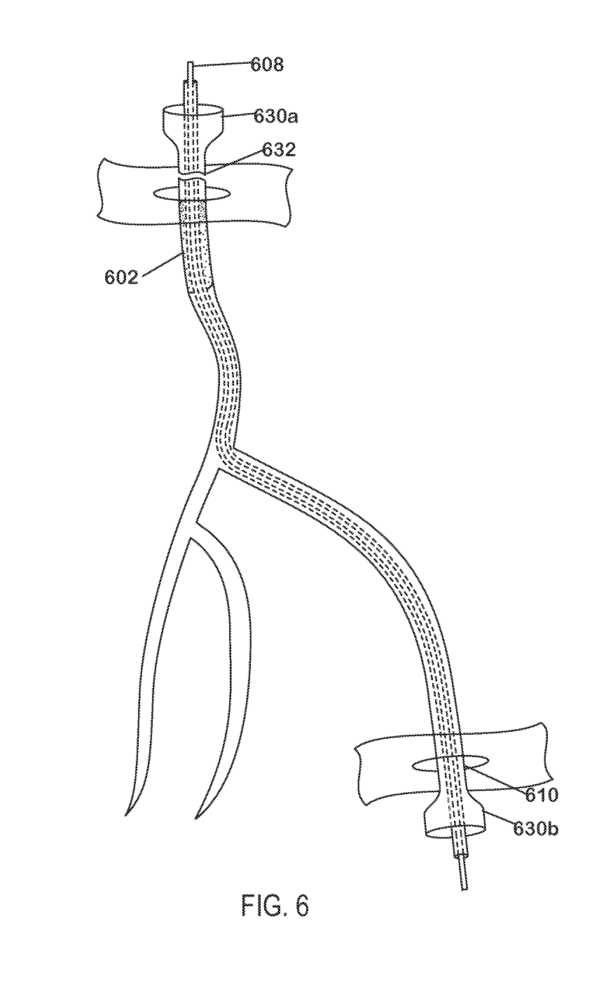

FIGS. 5A and 5B illustrate partial cutaway views of a peripheral artery in which another embodiment of a distal or retrograde approach to traversing a CTO is carried out, wherein a wire is advanced from the distal cap of the CTO through the proximal cap (FIG. 5A), where it is then captured by a capture wire using a neodymium magnet (FIG. 5B), in accordance with various embodiments. In the embodiment shown in FIGS. 5A and 5B, a recanalization wire 508 is advanced retrogradely through the anterior tibial artery or popliteal artery as described above until the distal cap 506 of the CTO 502 is reached. As described above, the recanalization wire 508 may then be advanced through the CTO 502 (either alone, as shown, or within a microcatheter, as described above with reference for FIGS. 4A and 4B) until the proximal cap 504 of the CTO 502 has been traversed. In various embodiments, the recanalization wire 508 may include a ferromagnetic tip 524.

Access to the femoral artery may be obtained through a puncture site in the groin, and a capture wire 520 may then be advanced anterogradely through the femoral artery as described above until it reaches the distal ferromagnetic tip 524 of the recanalization wire 508 at the proximal cap 504 of the CTO 502. In various embodiments, the distal tip of the capture wire 520 may include a neodymium magnet 526, thus the distal tip of the capture wire 520 may have a permanent magnetic charge that may have, for example, 10 times the strength of a naturally occurring magnet, or even more. In various embodiments, when the neodymium magnet 526 of the capture wire 520 makes contact with the ferromagnetic tip 524 of the recanalization wire 508, they couple together with a magnetic force having sufficient strength to allow the user to pull the recanalization wire 508 towards the aperture in the femoral artery, thus externalizing the wire. In various embodiments, this may create a complete traversal of the CTO 520 with a wire extending from the sheath in the femoral artery, through the CTO 502, and exiting through a sheath in the popliteal artery or the dorsalis pedis branch of the anterior tibial artery.

FIG. 7 illustrates a CTO in the superficial femoral artery, wherein the recanalization wire has traversed the CTO and is externalized through a first sheath in the dorsalis pedis branch of the tibial artery and a second sheath in the common femoral artery, in accordance with various embodiments. In the illustrated embodiment, once the CTO 602 has been completely traversed and the recanalization wire 608 has been externalized on both ends (e.g., via a first sheath 630a in the common femoral artery 632 and a second sheath 630b in the dorsalis pedis 610 or another artery in the lower leg or foot), the user may perform a procedure such as angioplasty or stenting in order to widen the passage through the CTO 602, thus treating the CTO 602 and restoring blood flow to the limb.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A method of traversing a chronic total occlusion (CTO), comprising:
advancing a recanalization wire through the vasculature of the subject through a puncture site in an artery of a lower extremity towards a distal cap of the CTO;
advancing the recanalization wire through the CTO until a distal tip of the recanalization wire exits the CTO via a proximal cap of the CTO;
magnetically capturing the distal tip of the recanalization wire; and
pulling the recanalization wire through a puncture side in a femoral artery, thus externalizing the recanalization wire.

2. The method of claim 1, wherein the artery of the lower extremity is a dorsalis pedis.

3. The method of claim 1, wherein the artery of the lower extremity is a posterior tibialis.

4. The method of claim 1, wherein the artery of the lower extremity is a peroneal (fibular) artery.

5. The method of claim 1, wherein the artery of the lower extremity is a popliteal artery.

6. The method of claim 1, wherein advancing the recanalization wire through the vasculature of the subject through a puncture site in an artery of the lower extremity comprises advancing the recanalization wire inside a microcatheter.

7. The method of claim 6, wherein advancing the recanalization wire through the CTO until the distal tip of the recanalization wire exits the CTO via the proximal cap of the CTO comprises advancing both the recanalization wire and the microcatheter through the CTO.

9. The method of claim 7, wherein the steps of (1) advancing the recanalization wire through the CTO; and (2) advancing the microcatheter through the CTO occur simultaneously.

10. The method of claim 1, wherein magnetically capturing the distal tip of the recanalization wire further comprises advancing a magnetic capture wire through the vasculature of the subject through a puncture site in a femoral artery towards the proximal cap of the CTO.

11. The method of claim 10, wherein the capture wire comprises a distal end portion, and wherein the distal end portion comprises a magnet.

12. The method of claim 11, wherein the magnet is a neodymium magnet.

13. The method of claim 11, wherein the distal tip of the recanalization wire comprises a ferromagnetic tip.

14. The method of claim 13, wherein capturing the distal tip of the recanalization wire with the capture wire comprises magnetically coupling the distal tip of the capture wire and the distal tip of the recanalization wire.

15. The method of claim 14, wherein magnetically coupling the distal tip of the capture wire and the distal tip of the recanalization wire comprises forming a magnetic coupling that is sufficiently strong to permit the recanalization wire to be pulled through the puncture side in a femoral artery.

16. The method of claim 1, wherein the method further includes performing an angioplasty, stenting procedure, or atherectomy on the CTO.
